(19) United States
(12) Patent Application Publication
(10) Pub. No.: US 2020/0268431 A1
(43) Pub. Date: Aug. 27, 2020

(54) RADIO FREQUENCY SURGICAL INSTRUMENTS

(71) Applicant: Medical Engineering Innovations, Inc., Madison, WI (US)

(72) Inventors: Richard Cary Schmidt, Verona, WI (US); Joshua Cook, Madison, WI (US); Steven Charles Southwell, Madison, WI (US); Leo R. Parpart, Madison, WI (US)

(21) Appl. No.: 16/803,504

(22) Filed: Feb. 27, 2020

Related U.S. Application Data
(60) Provisional application No. 62/811,136, filed on Feb. 27, 2019.

(51) Int. Cl. A61B 18/12

(52) U.S. Cl. CPC A61B 18/12; A61B 2018/00577

(57) ABSTRACT

The present invention relates to electrode arrays that find use for rapid ablation of a target area of tissue in an organ and in particular to use of the electrode arrays to resect organs to coagulate tissue so that resection can be performed with minimal loss of blood. The target area may include a bulk area of tissue (such as a tumor) or organ (such as a kidney) or a defined target area within a tissue or organ (such as a linear strip, curved strip, cylindrical area, or other shape).

### CROSS REFERENCE TO RELATED APPLICATIONS

This application claims the benefit of U.S. Provisional application Ser. No. 62/811,136 filed on Feb. 27, 2019, the entire contents of which are incorporated herein by reference.

### FIELD OF THE INVENTION

The present invention relates to electrode arrays that find use for rapid ablation of a target area in a tissue in an organ and in particular to use of the electrode arrays to coagulate tissue so that resection can be performed to resect organs with minimal loss of blood.

### BACKGROUND OF THE INVENTION

Liver resection is still the management of choice with the best chance for long-term cure in patients with primary and metastatic hepatic tumors. However, only a small percentage of these patients are candidates for curative surgical resection, because of the tumor size, location near major intrahepatic vessels, multifocality, or inadequate hepatic function related to coexistent cirrhosis. Traditionally, for a tumor to be considered appropriate for curative resection, there must not be any extrahepatic disease or severe hepatic dysfunction, the tumor or tumors must not be so extensive that too little functioning liver remains after the resection, and at least a 1-cm tumor-free resection margin should be attained and there should not be any involvement of the confluence of the portal vein.

Radio frequency (RF) ablation has become a widely used ablative technique for primary and secondary liver tumors and its safety, efficacy, and acceptable local recurrence and short-term survival rates had been well demonstrated in the literature. RF ablation is a technique based on the conversion of electromagnetic energy into heat to destroy tumors in various organs. It is either a useful adjunct therapy to partial liver resection or the primary modality of treatment for patients who were not candidates for curative resection.

Devices for use in RF ablation have been described. See, e.g., U.S. Pat. Nos. 7,367,974 and 10,130,415, both of which are incorporated herein by reference in their entirety. Some of the devices utilize an array of blades which are inserted into a target organ such as a liver. The tissue adjacent to the blades is coagulated, allowing resection of a portion of the organ. However, the blades can be quite large and thus cause trauma or excessive bleeding upon insertion.

What is needed in the art are ablation systems that minimize bleeding upon insertion into a target organ.

### SUMMARY OF THE INVENTION

The present invention relates to electrode arrays that find use for rapid ablation of a target area of tissue in an organ and in particular to use of the electrode arrays to resect organs to coagulate tissue so that resection can be performed with minimal loss of blood. The target area may include a bulk area of tissue (such as a tumor) or organ (such as an ovary) or a defined target area within a tissue or organ (such as a linear strip, curved strip, cylindrical area, or other shape).

Accordingly, in some embodiments, the present invention provides an ablation apparatus comprising: a radio frequency (RF) power source, a first set of two or more electrodes having tissue piercing distal portions, the first set of electrodes electrically connected via the RF power source, a second set of two or more electrodes having a tissue piercing distal portions, the second set of electrodes electrically connected via the RF power source, wherein the first and second set of electrodes are oriented so that when alternating current power is applied to either the first or second set of electrodes a current flows from that set of electrodes to the other of the first and second set of electrodes.

In some preferred embodiments, the electrodes are needle-shaped. In some preferred embodiments, the first and second set of electrodes each comprise from 2 to 10 electrodes. In some preferred embodiments, the first and second set of electrodes each comprise from 2 to 7 electrodes.

In some preferred embodiments, the first and second sets of electrodes are positioned in an electrically insulated holder. In some preferred embodiments, the first set of electrodes and the second set of electrodes are positioned in the electrically insulated holder so that the first set of electrodes is in a first substantially linear array and the second set of electrodes is in a second substantially linear array and wherein the first and second linear arrays are substantially parallel. In some preferred embodiments, the first set of electrodes and the second set of electrodes are positioned in the electrically insulated holder so that the first set of electrodes is in a first substantially linear array and the second set of electrodes is in a second substantially linear array and wherein the first and second linear arrays have angle therebetween of from 10 to 170 degrees. In some preferred embodiments, the first set of electrodes and the second set of electrodes each comprise three or more electrodes and sets of electrodes is positioned in the electrically insulated holder so that the first set of electrodes is in a first substantially curved array and the second set of electrodes is in a second substantially curved array. In some preferred embodiments, the first set of electrodes and the second set of electrodes each comprise three or more electrodes and the sets of electrodes are positioned in an electrically insulated holder so that the first set of electrodes is arranged in a non-linear array where one or more of the electrodes is offset from the other electrodes in the array and the second set of electrodes is arranged in a non-linear array where one or more of the electrodes is offset from the other electrodes in the array.

In some preferred embodiments, the ablation apparatus further comprises at least a third set of two or more electrodes having tissue piercing distal portions, the third set of electrodes electrically connected via the RF power source. In some preferred embodiments, the first set of electrodes, second set of electrodes and at least a third set of electrodes are positioned in an electrically insulated holder so that the first set of electrodes is in a first substantially linear array, the second set of electrodes is in a second substantially linear array, and the at least a third set of electrodes is in a third substantially linear array and wherein the first, second and third linear arrays are substantially parallel. In some preferred embodiments, the first set of electrodes, second set of electrodes and at least a third set of electrodes are positioned in an electrically insulated holder so that the first set of electrodes is in a first substantially linear array, the second set of electrodes is in a second substantially linear array, and the at least a third set of electrodes is in a third substantially linear array and wherein the first and second linear arrays have angle therebetween of from 10 to 170 degrees and the second and third linear arrays have angle therebetween of from 10 to 170 degrees. In some preferred embodiments, the first set of electrodes, second set of electrodes and at least a third set of electrodes each comprise three or more electrodes and the sets of electrodes are positioned in an electrically insulated holder so that the first set of electrodes is in a first curved array, the second set of electrodes is in a second curved array, and the at least a third set of electrodes is in a third linear array and wherein the first, second and third linear arrays are substantially parallel. In some preferred embodiments, the first set of electrodes, second set of electrodes and at least a third set of electrodes and the sets of electrodes are positioned in an electrically insulated holder so that the first set of electrodes is arranged in a non-linear array where one or more of the electrodes is offset from the other electrodes in the array, the second set of electrodes is arranged in a non-linear array where one or more of the electrodes is offset from the other electrodes in the array, and the at least a third set of electrodes is arranged in a non-linear array where one or more of the electrodes is offset from the other electrodes in the array.

In some preferred embodiments, the RF power comprises a switching circuit to allow sequential switching of current flow between the at least three sets of electrodes.

In some preferred embodiments, the sets of electrodes are movable between a collapsed position and an expanded position. In some preferred embodiments, the sets of electrodes movable between a collapsed position and an expanded position are arranged in hollow tube so that the sets of electrodes are collapsed when in the tube and expand when moved outside the tube. In some preferred embodiments, the hollow tube is a trocar. In some preferred embodiments, the hollow tube is a stent that is insertable into a luminal space in the body of a subject.

In some preferred embodiments, the RF is duty cycle modulated to control application of power to the organ. In some preferred embodiments, the RF power source comprises control circuits to control average current flow at the electrodes according to at least one parameter selected from the group consisting of: local temperature of the tissue, local impedance of the tissue, a predetermined current limit, and a predetermined power limit.

In some preferred embodiments, the present invention provides an ablation apparatus comprising: a radio frequency (RF) power source; a first set of two or more electrodes having tissue piercing distal portions, the first set of electrodes electrically connected via the RF power source; a second set of two or more electrodes having a tissue piercing distal portions, the second set of electrodes electrically connected via the RF power source; and a hollow tube, wherein the sets of electrode movable between a collapsed position and an expanded position and are arranged in the hollow tube so that the sets of electrodes are collapsed when in the tube and expand when moved to a position outside of the tube. In some preferred embodiments, the hollow tube is a trocar. In some preferred embodiments, the hollow tube is a stent that is insertable into a luminal space in the body of a subject.

In some preferred embodiments, the present invention provides methods of ablating a tissue comprising: providing a radio frequency (RF) power source, a first set of two or more electrodes having tissue piercing distal portions, the first set of electrodes electrically connected via the RF power source, and a second set of two or more electrodes having a tissue piercing distal portions, the second set of electrodes electrically connected via the RF power source; inserting said first and second set of electrodes into a tissue to be ablated; and applying alternating current power via said RF power source so that a current flows from that set of electrodes to the other of the first and second set of electrodes through the tissue thereby creating a zone of ablated tissue. In some preferred embodiments, the tissue is an organ and the zone of ablated tissue forms a partition across the organ and further comprising cutting the tissue of the organ at the partition of ablated tissue to reduce blood loss during resection of the organ. In some preferred embodiments, the partition of ablated tissue is positioned between the portion of the organ to be resected and a region of blood flow into the tissue.

In some preferred embodiments, the electrodes are needle-shaped. In some preferred embodiments, the first and second set of electrodes each comprise from 2 to 10 electrodes. In some preferred embodiments, the first and second set of electrodes each comprise from 2 to 7 electrodes.

In some preferred embodiments, the first and second sets of electrodes are positioned in an electrically insulated holder. In some preferred embodiments, the first set of electrodes and the second set of electrodes are positioned in the electrically insulated holder so that the first set of electrodes is in a first substantially linear array and the second set of electrodes is in a second substantially linear array and wherein the first and second linear arrays are substantially parallel. In some preferred embodiments, the first set of electrodes and the second set of electrodes are positioned in the electrically insulated holder so that the first set of electrodes is in a first substantially linear array and the second set of electrodes is in a second substantially linear array and wherein the first and second linear arrays have angle therebetween of from 10 to 170 degrees. In some preferred embodiments, the first set of electrodes and the second set of electrodes each comprise three or more electrodes and sets of electrodes is positioned in the electrically insulated holder so that the first set of electrodes is in a first substantially curved array and the second set of electrodes is in a second substantially curved array. In some preferred embodiments, the first set of electrodes and the second set of electrodes each comprise three or more electrodes and the sets of electrodes are positioned in an electrically insulated holder so that the first set of electrodes is arranged in a non-linear array where one or more of the electrodes is offset from the other electrodes in the array and the second set of electrodes is arranged in a non-linear array where one or more of the electrodes is offset from the other electrodes in the array.

In some preferred embodiments, the ablation apparatus further comprises at least a third set of two or more electrodes having tissue piercing distal portions, the third set of electrodes electrically connected via the RF power source. In some preferred embodiments, the first set of electrodes, second set of electrodes and at least a third set of electrodes are positioned in an electrically insulated holder so that the first set of electrodes is in a first substantially linear array, the second set of electrodes is in a second substantially linear array, and the at least a third set of electrodes is in a third substantially linear array and wherein the first, second and third linear arrays are substantially parallel. In some preferred embodiments, the first set of electrodes, second set of electrodes and at least a third set of electrodes are positioned in an electrically insulated holder so that the first set of electrodes is in a first substantially linear array, the second set of electrodes is in a second substantially linear array, and the at least a third set of electrodes is in a third substantially linear array and wherein the first and second linear arrays have angle therebetween of from 10 to 170 degrees and the second and third linear arrays have angle therebetween of from 10 to 170 degrees. In some preferred embodiments, the first set of electrodes, second set of electrodes and at least a third set of electrodes each comprise three or more electrodes and the sets of electrodes are positioned in an electrically insulated holder so that the first set of electrodes is in a first curved array, the second set of electrodes is in a second curved array, and the at least a third set of electrodes is in a third linear array and wherein the first, second and third linear arrays are substantially parallel. In some preferred embodiments, the first set of electrodes, second set of electrodes and at least a third set of electrodes and the sets of electrodes are positioned in an electrically insulated holder so that the first set of electrodes is arranged in a non-linear array where one or more of the electrodes is offset from the other electrodes in the array, the second set of electrodes is arranged in a non-linear array where one or more of the electrodes is offset from the other electrodes in the array, and the at least a third set of electrodes is arranged in a non-linear array where one or more of the electrodes is offset from the other electrodes in the array.

In some preferred embodiments, the RF power comprises a switching circuit to allow sequential switching of current flow between the at least three sets of electrodes.

In some preferred embodiments, the sets of electrodes are movable between a collapsed position and an expanded position. In some preferred embodiments, the sets of electrodes movable between a collapsed position and an expanded position are arranged in hollow tube so that the sets of electrodes are collapsed when in the tube and expand when moved outside the tube. In some preferred embodiments, the hollow tube is a trocar. In some preferred embodiments, the hollow tube is a stent that is insertable into a luminal space in the body of a subject.

In some preferred embodiments, the RF is duty cycle modulated to control application of power to the organ. In some preferred embodiments, the RF power source comprises control circuits to control average current flow at the electrodes according to at least one parameter selected from the group consisting of: local temperature of the tissue, local impedance of the tissue, a predetermined current limit, and a predetermined power limit.

In some preferred embodiments, the present invention provides an apparatus as described above for use in ablating tissue. In some preferred embodiments, the tissue in an organ in need of resection. In some preferred embodiments, ablation of tissue within the organ creates a partition positioned between the portion of the organ to be resected and a region of blood flow into the tissue.

### BRIEF DESCRIPTION OF THE DRAWINGS

FIG. 1 is a perspective view of an electrode array of the present invention.

FIG. 2 is schematic diagram of an ablation apparatus of the present invention showing electrical fields and equipotential lines for a virtual two-blade array.

FIG. 3 is a schematic diagram showing electrical fields and equipotential lines for a virtual “three-blade” array.

FIG. 4 is a block diagram of an RF power supply suitable for use with electrode arrays of the present invention.

FIG. 5 is a perspective view of an additional embodiment of an electrode array of the present invention.

FIG. 6 is a schematic diagram of electrical fields and equipotential lines from a virtual two-blade array where the blades are positioned at 90° to one another.

FIG. 7 is a schematic diagram of electrical fields and equipotential lines from a virtual three-blade array where the blades are positioned at 45° to one another.

FIG. 8 is a block diagram of RF generators attached to a virtual four-blade array.

FIG. 9 is a perspective view of an additional embodiment of an electrode array of the present invention.

FIG. 10 is a schematic diagram of electrical fields and equipotential lines from a virtual two-blade array where the virtual blades are curved.

FIG. 11 is a perspective view of an additional embodiment of an electrode array of the present invention.

FIG. 12 is a schematic diagram of electrical fields and equipotential lines from a virtual two-blade array.

FIG. 13 is a schematic diagram of electrical fields and equipotential lines from a virtual two-blade array.

FIGS. 14A, 14B, 14C and 14D provide cross-sectional views of an expandable two electrode array of the present invention in retracted (FIG. 14A), intermediate (FIG. 14B), fully expanded (FIG. 14C) and treatment (FIG. 14D) positions.

FIGS. 15A, 15B and 15C provide cross-sectional views of an expandable four electrode array of the present invention in retracted (FIG. 15A), intermediate (FIG. 15B) and fully expanded (FIG. 15C) positions.

FIGS. 16A and 16B provide end views of an expandable two electrode array set (FIG. 16A) and four electrode array set (FIG. 16B).

FIGS. 17A, 17B and 17C provide block diagrams depicting connection of an expandable two electrode array set (FIG. 17A) and expandable four electrode array sets (FIG. 17B and FIG. 17C).

### DETAILED DESCRIPTION OF THE INVENTION

The present invention relates to electrode arrays that find use for rapid ablation of a target area of tissue in an organ and in particular to use of the electrode arrays to resect organs to coagulate tissue so that resection can be performed with minimal loss of blood

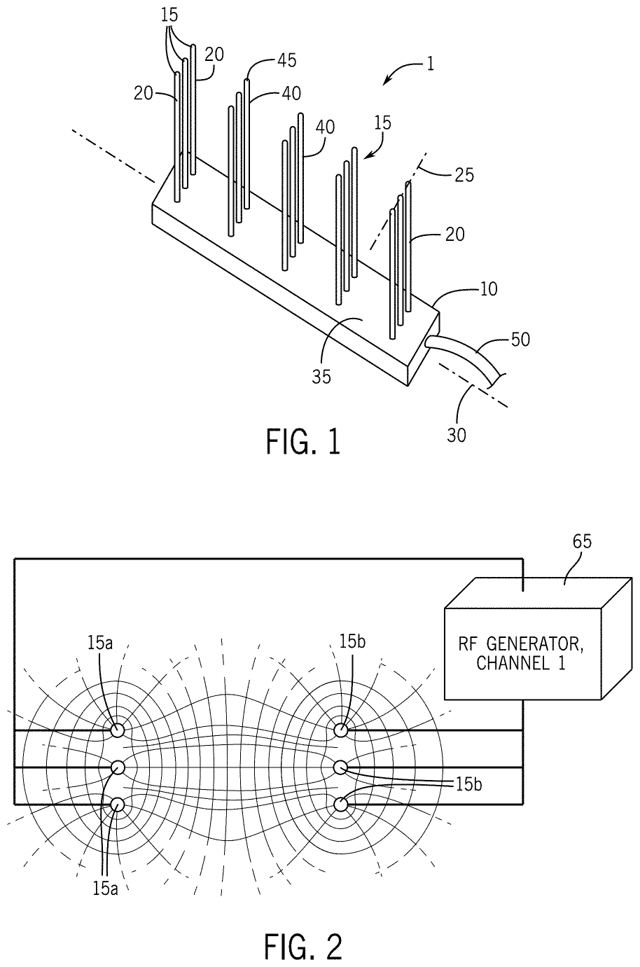

Referring to FIG. 1, an electrode array assembly 1 of the present invention includes a holder 10 supporting two or more of sets of elongate electrodes 15 each comprising a plurality of individual elongate electrodes 20. The present invention is not limited to any particular arrangement of the sets of electrodes or individual electrodes. In FIG. 1, each set of elongate electrodes 15 in the holder 10 defines a linear probe axis 25 and the sets of the elongate electrodes 15 are substantially parallel to one another (i.e., the linear probe axes are substantially parallel). In some preferred embodiments, the sets of elongate electrodes are spaced along an electrode set axis 30. In some preferred embodiments, the sets of elongate electrodes define a generally planar surface 35. A variety of other preferred embodiments for arrangement of electrode sets are provided below.

The individual elongate electrodes 20 preferably have a tissue piercing distal portion 40 such as a sharpened tip 45. The elongated electrodes 20 may therefore be inserted into an organ, for example a liver, to isolate a portion of the organ for resection. The elongate electrodes 20 may, for example, be constructed of biocompatible stainless steel or other suitable materials. In some preferred embodiments, the elongate electrodes 20 may preferably be from 1 to 20 cms in length and from 0.5 to 3 mm in diameter. The present invention is not limited to any particular number of individual elongated electrodes 20 that are included within a set of elongated electrodes 15. For example, a set of elongated electrodes 15 may preferably comprise 2, 3, 4, 5, 6, 7, 8, 9, 10 or up to 20, 30 or 40 individual elongated electrodes.

The holder 10 may be, for example, an insulating material block having holes cut in the holder 10 to receive metallic shafts of the elongate electrodes 20 at regular intervals. In a preferred embodiment, the separation of the sets of electrodes 15 is from 0.5 to 2.0 cm. The elongate electrodes 20 may be fixed to the holder 10 so as to be moved in unison for rapid insertion. In some preferred embodiments, each set of elongate electrodes 15 may be independently attached to a separate conductor (not shown in FIG. 1) of a cable 50 providing independently controllable RF power to each of the sets of elongate electrodes 15 as will be described below. The holder may be any size or dimension suitable for application to a target organ or region of a target organ. For example, the holder 10 may be from 2 cm to 20 cm along the electrode set axis 30. Holder 10 may be made of a material that is flexible or hinged in one or more planes in order to allow the user to dynamically change the geometry of the electrode array, for example, a linear array may be bent slightly in the plane of the large surface of block 10 in order to avoid a critical anatomical structure that must remain intact. Additionally, block 10 may be deformed/hinged out of plane in order to accommodate the shape of a structure, such as a roughly spherical tumor, which the user desires to treat. Another embodiment may use more than one block 10. For example, one electrode set is contained in one block 10 and the second electrode set is attached to the other block 10. This would allow the user to position the electrode sets independently to achieve the desired result.

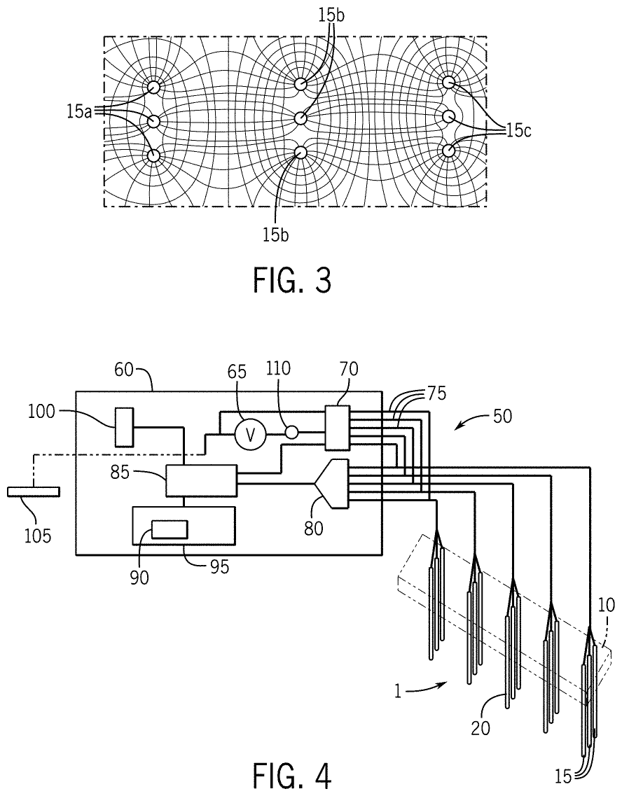

It will be understood that the blades of current RF resection devices such as those disclosed in U.S. Pat. No. 7,367,974 (incorporated herein by reference in its entirety) may be replaced by sets or arrays of electrically connected needle-shaped electrodes as depicted in FIGS. 1-3. These needle-shaped electrodes do not need to have a circular cross-section, but could be square, rectangular, or any other desirable cross-section shape. It is contemplated that each electrically connected set 15 of individual electrodes 20 is “virtually” equivalent in shape to a blade-shaped electrode when power is applied to the electrode sets as depicted in FIGS. 2 and 3. Referring to FIG. 2, each set of electrodes 15a and 15b is preferably to a power source comprising a radiofrequency (RF) generator 65. In preferred embodiments, each individual electrode 20 (end view) within an electrode set 15 (end view) is electrically connected to the other electrodes within the set so that current flows between the sets of electrodes as depicted in FIG. 2. Three individual electrodes are shown for each “virtual electrode” or set of elongated electrodes 15. However, the present invention is not limited to this particular arrangement of number of individual electrodes within a set. FIG. 2 further provides a schematic depiction of the electric fields and equipotential lines around sets of closely spaced individual needle electrodes. The volume between the electrode sets, the electric fields, and the equipotential lines are essentially the same as those formed by parallel plate (i.e., blade-shaped) electrodes. FIG. 3 provides a simulation of the electric fields and equipotential lines for a “virtual three blade” array comprising three sets of elongated electrodes 15a, 15b and 15c. Connection with the RF generator and switching of power between sets of electrodes is described in more detail below.

In preferred embodiments, the electrode sets are electrically connected so that the electric fields formed are essentially the same as those formed by larger electrodes. The sets of electrodes as depicted cause much less trauma and bleeding when inserted into an organ as compared to blade-shaped electrodes. In particular, the present invention provides substantial improvements because the use of sets of elongated probes of small diameter as disclosed in the present invention provides the lowest risk to blood vessels during insertion while providing fast, uniform coagulation that is characteristic of larger blade-shaped electrodes.

Referring now to FIG. 4, the electrode array assembly 1 may be used in conjunction with a power unit 60 providing an RF power source 65. The power unit 60 provides power to the sets of elongate electrodes 15 via an electronically controllable switching circuit 70 communicating with the multiple conductors 75 of cable 50 (or cables) passing to the elongate electrode sets 15. RF power sources 65 suitable for multiple electrodes are described in U.S. application Ser. No. 10/796,239 filed Mar. 9, 2004 and entitled Multipolar Electrode System for Volumetric Radio Frequency Ablation and U.S. application Ser. No. 10/11,681 filed Jun. 10, 2002 and entitled: Radio-Frequency Ablation System Using Multiple Electrodes, both hereby incorporated by reference in their entirety.

The power unit 60 may also receive signals from each of the sets elongate electrodes 15 from an optional thermal sensors (not shown), such as a thermocouple or solid-state temperature sensor, attached to the surface of the elongate electrodes 20 or within the electrodes. Signals from these thermal sensors may be received by the power unit at input circuit 80 which digitizes and samples the temperature signals and provides them to a microprocessor 85. In a similar fashion, the power unit may receive voltage and current measurements from each electrode set and provide feedback based on tissue impedance. In still other embodiments, the amount of power provided to electrode sets is monitored.

The microprocessor 85 executes a stored program 90 held in a memory 95 and also communicates with a front panel control set 100 to provide data to a user and accept user input commands.

While the present invention contemplates that power will be applied to the sets of elongate electrodes 15 in a bipolar mode as will be described, power unit 60 may alternatively communicate with a ground pad 105 to allow monopolar operation.

The switching circuit 70 provides switches that allow each conductor 75 attached to a set of elongate electrodes 15 to be switched to either terminal of the RF power source 65 so that the set of elongate electrodes 15 provides either a return or source of RF power. Switching circuit 70 may also be used to disconnect particular ones of the conductors 75 so as to isolate the associated set of elongate electrodes 15 and to allow a duty cycle modulated control of the power going to each set of elongate electrodes 15. Thus, while the power source 65 may optionally run at a constant rate control of the power may be obtained through the switching circuit 70. It is desirable that switching of RF circuits occur only at the time that the voltage crosses zero volts. This is commonly known as “zero-crossing”. In preferred embodiments, the switching circuit 70 is connected to the microprocessor 85 to be controlled thereby. For bipolar operation, each RF channel has two conductors. The voltage on a single RF conductor oscillates from −V to +V repeatedly. The two conductors from a single RF channel are out of phase so that when one is at +V, the other is at −V. The switching circuit can be used to supply RF power to multiple pairs of electrodes in sequence from a single channel RF generator, and it can be used to connect and disconnect the RF power to electrode pairs.

The microprocessor 85 in a preferred embodiment executes the program 90 in memory 95 to sequentially control the switches of the switching circuit 70 to connect one pair of sets of elongate electrodes 15 to the power source 70 at each time. Accordingly, at a time period 1, a pair of sets of elongate electrodes will be connected across power source 65 for current to flow therebetween. At this time, all other sets of elongate electrodes 15 are disconnected from the power source 65. At a second time period 2, a second pair of sets of elongate electrodes 15 will be connected across the power source 65 for power to flow therebetween and a previously utilized set of elongate electrodes is disconnected from the power source 65. In other embodiments, the power units incorporate a separate RF power source for each pair of electrodes so that all volumes of tissue may be energized at the same time. In still other preferred embodiments, multiple RF power sources are provided in the power unit and are used to energize pairs of electrodes sequentially.

This process repeats itself for the remaining sets of elongate electrodes 15 until each electrode has been pair-wise connected to the power source 65. After this, the cycle is reinitiated.

In an alternative embodiment, each of the sets of elongate electrodes 15 other than the pair being connected to the power source 65 is connected to a return path so as to provide an effective virtual ground plane for return of current. In monopolar operation, only one set of electrodes is connected to the RF power source (as opposed to pairs) and the other connection of the RF power source is to a ground.

In yet another alternative embodiment, the sequential switching of pairs of sets elongate electrodes 15 does not proceed continuously from left to right but rather every other sequential pairing is skipped to allow cooling of the tissue near each energized electrode before the next adjacent pair is energized. Accordingly, a first pair of a set of elongate electrodes may be connected across the power source 65 and then a separate second pair, and then a separate third pair, and so forth.

As well as limiting the overheating of tissue, the switching of the sets of elongate electrodes 15 provides other benefits. A large number of sets of elongate electrodes 15 may create a very low impedance device which may be beyond the current capability of standard power sources 65. Accordingly, the switched operation also allows that power to be allocated among pairs of the sets of elongate electrodes 15. With standard power sources 65, the ablation region will typically be 1 to 2 cm wide and can be obtained in five to ten minutes. The switching among sets of elongate electrodes 15 may also eliminate shielding effects among electrodes providing a more uniform ablation region.

The amount of power deposited at the tissue surrounding each set of elongate electrodes 15 may be changed by varying the length of the duration for which the sets of elongated electrodes are energized. Alternatively, a high-frequency duty cycle modulation may be imposed on the power applied across the sets of elongated electrodes according to well-known techniques.

The control of power deposited at the tissue near each set of elongated electrodes 15 may be controlled by these techniques according to the temperature measured at each set of elongate electrodes 15, for example, to reduce power when the temperature rises above a pre-determined threshold either according to a simple thresholding technique or a more complex feedback loop using proportional, integral, and derivative terms.

As an alternative to temperature control, the impedance of the tissue between each pair of a set of elongated electrodes 14 may be determined by monitoring the current flow into the tissue and the particular voltage of the power source 65 (using an in-line current sensor 110), and this impedance can be used to control power by decreasing, or shutting down power for a certain time period as impedance rises, the latter indicating a heating of the tissue.

Impedance measurements can also be used to gauge the thickness of the tissue being ablated. The tissue may have different thickness in the slice where the electrode array assembly 1 is inserted. By measuring impedance (with low power application of RF current) between adjacent sets of elongated electrodes 15, the slice thickness along the electrodes 15 can be estimated before ablating the slice. Power applied between each electrode pair can then be applied according to tissue thickness (e.g. tissue twice as thick requires approximately twice the power). In one embodiment, this can be achieved by applying a constant voltage bipolar between each electrode pair. If tissue is twice as thick, impedance is about half as great, and as a result the applied power is twice as high with that constant voltage.

Monitoring current and voltage with the microprocessor 85 may also be used to detect excess or low currents to any particular set of elongate electrodes 15. In the former case, power limiting may be imposed. The latter case may indicate a disconnection of one or more sets of elongate electrodes 15 and an indication of this may be provided on the front panel control set 100 to the user.

It will be apparent to those of ordinary skill in the art that a number of other control feedback techniques may be used including those which control current flow or voltage or power (the latter being the product of current and voltage) according to each of these terms.

As mentioned above, the present invention is not limited to devices with any particular arrangement of elongated electrodes 20 or sets of elongated electrodes 15.

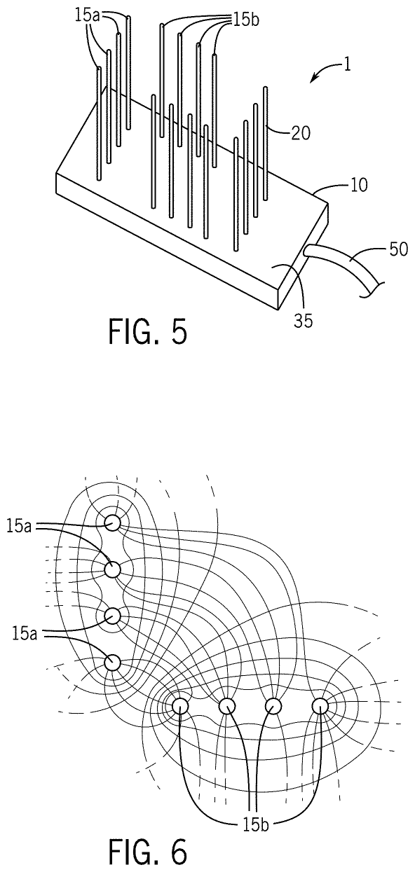

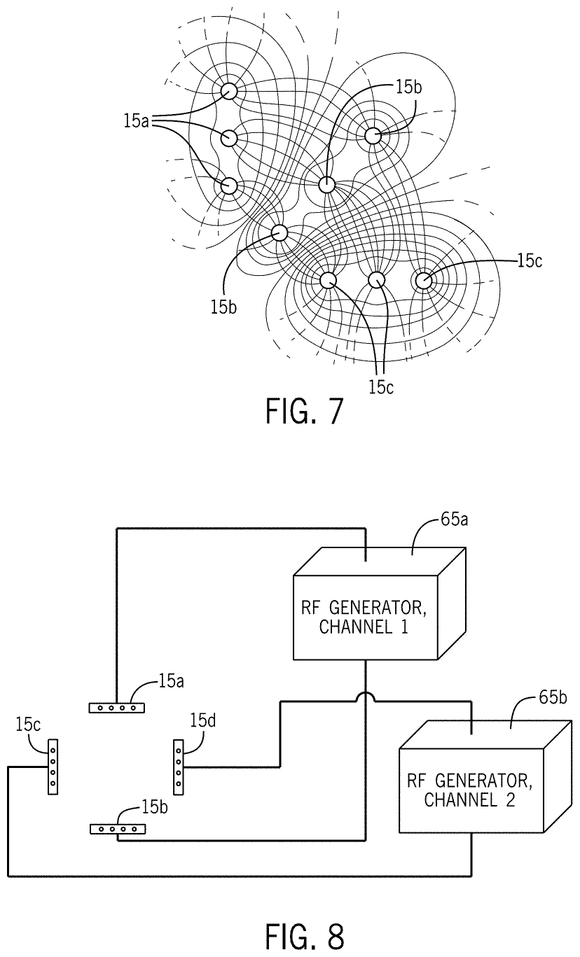

Referring to FIG. 5, in some preferred embodiments, the electrode array assembly 1 of the present invention includes two or more of sets of elongate electrodes 15 each comprising a plurality of individual elongate electrodes 20 so that at least a pair of the sets of electrodes are at an angle to one another. The sets of electrodes 15 may preferably be supported by holder 10. The sets of electrodes 15 are preferably connected to a power source as described above via cable 50. In the depicted embodiment, elongated electrode set 15a is at an angle of about 90° as compared to elongated electrode set 15b. However, the present invention is not limited to any particular angle between sets of elongated electrodes. The angle between sets of electrodes 15 within a pair (e.g., 15a and 15b) may be varied, for example, the range of useful angles may be from 10° to 170°, 15° to 75°, 30° to 90°, 60° to 120°, or 90° to 150°. FIG. 6 provides a schematic depicting the electric fields and equipotential lines for a pair of sets of elongated electrodes 15a and 15b that are oriented at approximately 90° to one another. FIG. 7 provides a schematic depicting the electric fields and equipotential lines for a three sets of elongated electrodes 15a, 15b and 15c that are oriented at approximately 45° to one another.

It will also be apparent that the present invention encompasses embodiments where different sets of elongated electrodes are paired in varying ways. For example, FIG. 8 provides a schematic depicting four sets of elongated probes 15a, 15b, 15c, and 15d, which can be provided in holder 10 (not shown) as described above. In the depicted embodiment, sets of elongated electrodes 15a and 15b are paired via a first channel of an RF generator 65a and electrodes 15c and 15d are paired via a second channel of an RF generator 65b. In this embodiment, the first pair of sets of elongated electrodes 15a and 15b are parallel to one another and the second pair of sets of elongated electrodes 15c and 15d are also parallel to one another. It will be readily understood that in another embodiment, sets of elongated electrodes 15a and 15d could be paired and that sets of elongated electrodes 15b and 15c could be paired.

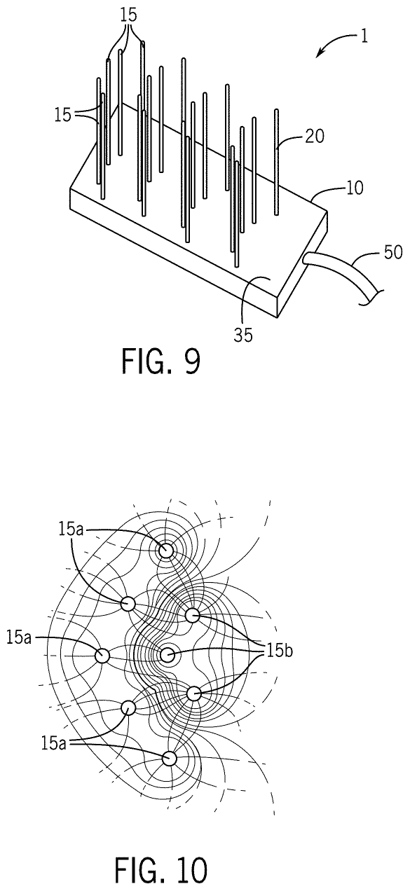

Referring to FIG. 9, in other preferred embodiments, the electrode array assembly 1 of the present invention includes two or more of sets of elongate electrodes 15 each comprising a plurality of individual elongate electrodes 20 so that the individual electrodes 20 within a set of elongated electrodes 15 define a curve. The curved sets of electrodes 15 may preferably be supported by holder 10. The curved sets of electrodes 15 are preferably connected to a power source as described above via cable 50. In the depicted embodiment, the curved sets of elongated electrodes are oriented along the long axis of the holder 10, but it will be recognized that pairs of the curved sets of electrodes 15 could be set at angles to one another as described above. FIG. 10 provides a schematic depicting the electric fields and equipotential lines for a pair of a curved set of elongated electrodes 15a and 15b. It is important to note, as shown in FIG. 10 that pairs of electrode sets do not need to be comprised of the same number of individual electrodes, or even comprised of electrodes that have the same cross-sectional shape. Other electrode geometries may be used in order to create the desired coagulation or ablation zone geometry.

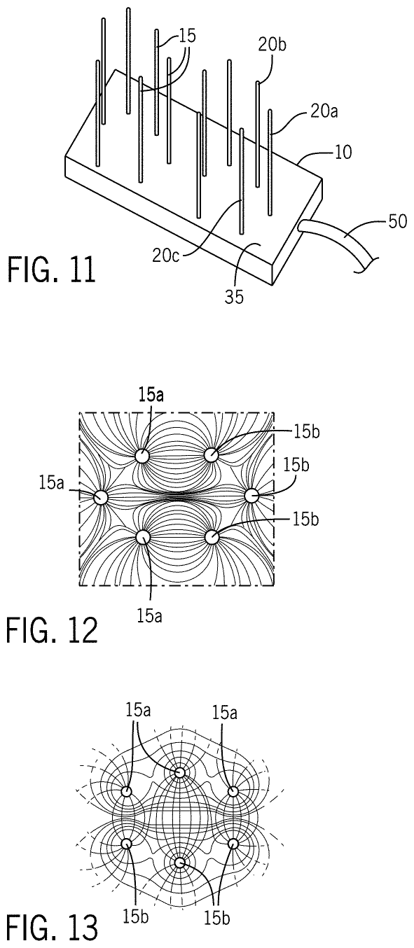

Referring to FIG. 11, in still other preferred embodiments, the electrode array assembly 1 of the present invention includes two or more of sets of elongate electrodes 15 each comprising a plurality of individual elongate electrodes 20 so that at least one individual electrodes 20a within a set of elongated electrodes 15 is offset from a line established by two or more electrodes 20b and 20c of the set of elongated electrodes 15. The offset sets of electrodes 15 may preferably be supported by holder 10. The offset sets of elongated electrodes 15 are preferably connected to a power source as described above via cable 50. In the depicted embodiment, the offset sets of elongated electrodes are oriented along the long axis of the holder 10, but it will be recognized that pairs of the offset sets of electrodes 15 could be set at angles to one another as described above. FIGS. 12 and 13 provide a schematic depicting the electric fields and equipotential lines for pairs of offset set of elongated electrodes 15a and 15b.

It will further be appreciated that various electrode array assemblies described above could be further customized by varied pairing of elongated electrodes or sets of elongated electrodes within an array. The different electrodes or sets of electrodes are preferably paired by switching electrical connections from one or more RF generators to designated pairs of electrode sets to provide electrical fields and thus coagulation zones of any shape, position or size within the needle array. Preferably, the designated electrode sets are designated via a computer interface such as a touch screen or by mechanically inserting and/or removing sets of elongated electrodes in a holder.

In still other embodiments, the sets of elongated electrodes are configured to be movable between a collapsed position and an expanded position. For example, such expandable electrode arrays are provided in a hollow tube such as in a trocar, stent or laparoscopic device for insertion into an organ or luminal space where ablation is desired. The arrays are preferably movable between a collapsed position inside the hollow tube and an expanded position when outside of the hollow tube.

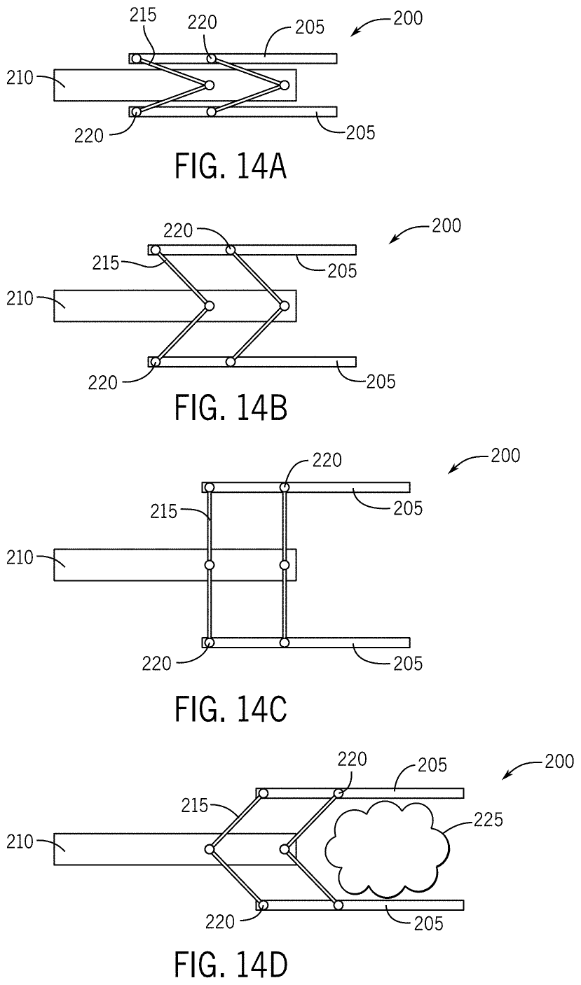

FIGS. 14a, 14b, 14c, and 14d provide a cross sectional view of an expandable electrode array 200 of the present invention. These figures depict an expandable set of elongated electrodes. It will be readily understood that multiple expandable sets of elongated electrodes can be provided, for example, in a stent, trocar, or laparoscope. In the depicted embodiments, the individual electrodes 205 are connected to an RF power source as described above. It will be understood that individual electrodes 205 can be replaced by sets of elongated electrode sets as described above, also attached to an RF power source as described above. FIG. 14A depicts an expandable set of elongated electrodes in a collapsed position. The individual electrodes 205 are attached to a support rod 210 via linkage arms 215 at attachment points 220. FIGS. 14B and 14C depict the expandable set of elongated electrodes to an intermediate position (FIG. 14B) and fully expanded position (FIG. 14C) during expansion by actuation of the linkage arms. FIG. 14d depicts the expandable set of electrodes after moving past the fully expanded position to a treatment position and surrounding a target tissue such as a tumor 225. Actuation of expansion is preferably via cable, springs, expanding material, memory material, or the like. Preferably the arrays may be both extended from the hollow tube to move to the expanded position and retracted into the hollow tube to move to a collapsed position. Moving the electrodes past the fully expanded configuration allows them to be placed close or in contact with the tissue to be treated (FIG. 14D). In this embodiment, RF or microwave electrodes do not need to penetrate the tissue, which removes the risk of releasing and re-seeding malignant cells elsewhere in the body. Again, only two electrodes in the array are shown, but other numbers of electrodes may be utilized in the array.

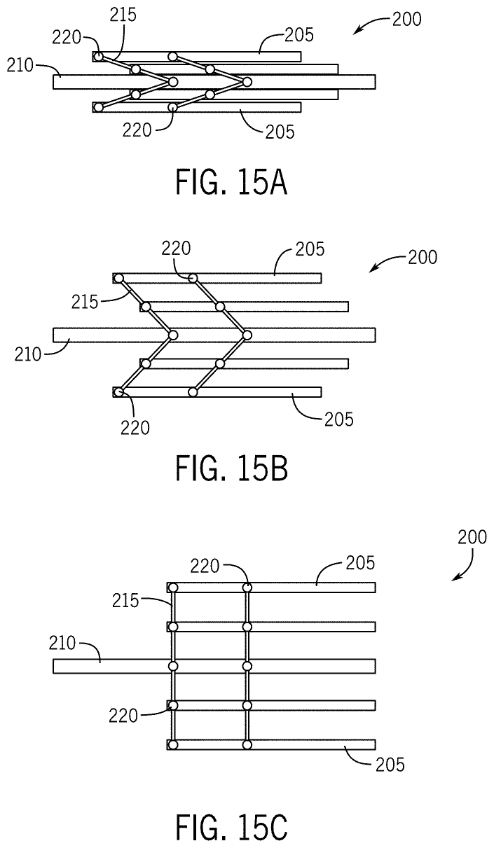

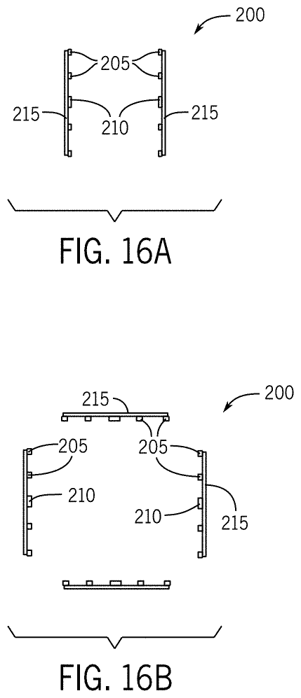

FIGS. 15A, 15B, 15C, and 15D depict another expandable set of elongated electrodes comprising five elongated electrodes 205 attached to a support rod 210 via linkage arms 215 as shown in FIG. 15A. FIGS. 15B and 15C depict the expandable set of elongated electrodes to an intermediate position (FIG. 14B) and fully expanded position (FIG. 14C) during expansion by actuation of the linkage arms. As above, only five electrodes in the array are shown, but other numbers of electrodes may be utilized in the array and they may be organized into sets of elongated electrodes as described in detail above. For example, FIGS. 16A and 16B provide an end view of two sets (FIG. 16A) and four sets (FIG. 16B) of elongated electrodes in an expanded position. In these embodiments, the support rod is also an electrode.

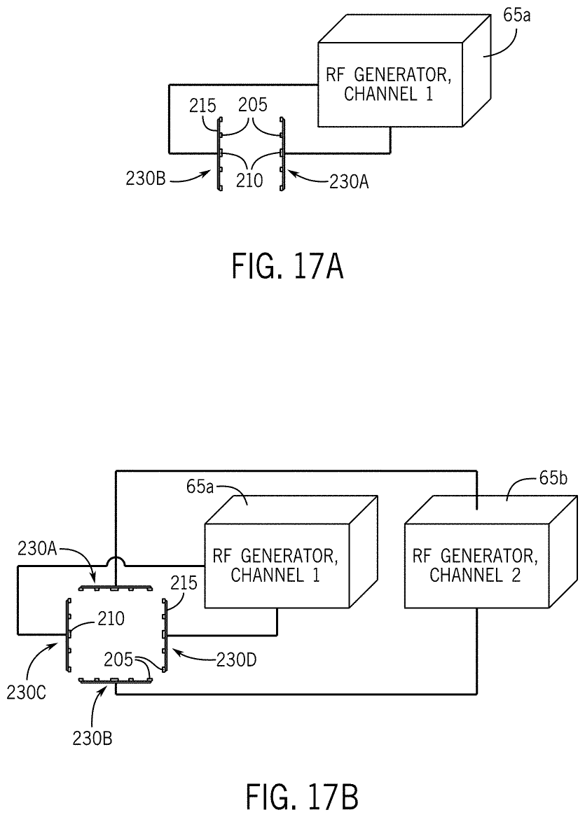

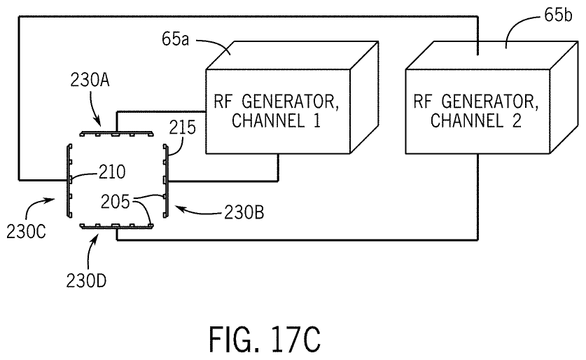

These sets of elongated electrodes may be connected to an RF generator as described above with respect to the sets of elongated electrodes provided in a holder. Exemplary connection embodiments are provided in FIGS. 17A, 17B and 17C. FIG. 17A provides a schematic depicting two expandable sets of elongated probes 230a and 230b paired via a first channel of an RF generator 65a. FIG. 17B provides a schematic depicting four expandable sets of elongated probes 230a, 230b, 230c, and 230d. In the depicted embodiment, sets of elongated electrodes 230a and 230b are paired via a first channel of an RF generator 65a and electrodes 230c and 230d are paired via a second channel of an RF generator 65b. In this embodiment, the first pair of sets of elongated electrodes 230a and 230b are parallel to one another and the second pair of sets of elongated electrodes 230c and 230d are also parallel to one another. FIG. 17C provides a schematic depicting four expandable sets of elongated probes 230a, 230b, 230c, and 230d. In the depicted embodiment, sets of elongated electrodes 230a and 230b are paired via a first channel of an RF generator 65a and electrodes 230c and 230d are paired via a second channel of an RF generator 65b. In this embodiment, the first pair of sets of elongated electrodes 230a and 230b are at 90° angles to one another and the second pair of sets of elongated electrodes 230c and 230d are also at 90° angles to one another.

The present invention is not limited to use with the liver, but may be used generally in any medical procedure where a barrier needs to be created prior to a cutting of tissue and in particular for surgery in other organs, or where a section of tissue needs to be destroyed by thermal ablation. The switching schedule through which power deposition is controlled may be regular or varied. The devices of the present invention are also useful for ablation of target tissues such as tumors and organs such as a uterus or kidney.

It is specifically intended that the present invention not be limited to the embodiments and illustrations contained herein, but include modified forms of those embodiments including portions of the embodiments and combinations of elements of different embodiments as come within the scope of the following claims. For example, the range of RF frequencies used in the present invention may extend from the kilohertz range to microwave frequencies using appropriate electrode structures.

What is claimed is:

1. An ablation apparatus comprising:
a radio frequency (RF) power source,
a first set of two or more electrodes having tissue piercing distal portions, the first set of electrodes electrically connected via the RF power source
a second set of two or more electrodes having a tissue piercing distal portions, the second set of electrodes electrically connected via the RF power source
wherein the first and second set of electrodes are oriented so that when alternating current power is applied to either the first or second set of electrodes a current flows from that set of electrodes to the other of the first and second set of electrodes.

2. The ablation apparatus of claim 1, wherein the electrodes are needle-shaped.

3. The ablation apparatus of claim 1, wherein the first and second set of electrodes each comprise from 2 to 10 electrodes.

4. The ablation apparatus of claim 1, wherein the first and second sets of electrodes are positioned in an electrically insulated holder.

5. The ablation apparatus of claim 4, wherein the first set of electrodes and the second set of electrodes are positioned in the electrically insulated holder so that the first set of electrodes is in a first substantially linear array and the second set of electrodes is in a second substantially linear array and wherein the first and second linear arrays are substantially parallel.

6. The ablation apparatus of claim 4, wherein the first set of electrodes and the second set of electrodes are positioned in the electrically insulated holder so that the first set of electrodes is in a first substantially linear array and the second set of electrodes is in a second substantially linear array and wherein the first and second linear arrays have angle therebetween of from 10 to 170 degrees.

7. The ablation apparatus of claim 4, wherein the first set of electrodes and the second set of electrodes each comprise three or more electrodes and sets of electrodes is positioned in the electrically insulated holder so that the first set of electrodes is in a first substantially curved array and the second set of electrodes is in a second substantially curved array.

8. The ablation apparatus of claim 4, wherein the first set of electrodes and the second set of electrodes each comprise three or more electrodes and the sets of electrodes are positioned in an electrically insulated holder so that the first set of electrodes is arranged in a non-linear array where one or more of the electrodes is offset from the other electrodes in the array and the second set of electrodes is arranged in a non-linear array where one or more of the electrodes is offset from the other electrodes in the array.

9. The ablation apparatus of claim 1, further comprising at least a third set of two or more electrodes having tissue piercing distal portions, the third set of electrodes electrically connected via the RF power source.

10. The ablation apparatus of claim 9, wherein the first set of electrodes, second set of electrodes and at least a third set of electrodes are positioned in an electrically insulated holder so that the first set of electrodes is in a first substantially linear array, the second set of electrodes is in a second substantially linear array, and the at least a third set of electrodes is in a third substantially linear array and wherein the first, second and third linear arrays are substantially parallel.

11. The ablation apparatus of claim 9, wherein the first set of electrodes, second set of electrodes and at least a third set of electrodes are positioned in an electrically insulated holder so that the first set of electrodes is in a first substantially linear array, the second set of electrodes is in a second substantially linear array, and the at least a third set of electrodes is in a third substantially linear array and wherein the first and second linear arrays have angle therebetween of from 10 to 170 degrees and the second and third linear arrays have angle therebetween of from 10 to 170 degrees.

12. The ablation apparatus of claim 9, wherein the first set of electrodes, second set of electrodes and at least a third set of electrodes each comprise three or more electrodes and the sets of electrodes are positioned in an electrically insulated holder so that the first set of electrodes is in a first curved array, the second set of electrodes is in a second curved array, and the at least a third set of electrodes is in a third linear array and wherein the first, second and third linear arrays are substantially parallel.

13. The ablation apparatus of claim 9, wherein the first set of electrodes, second set of electrodes and at least a third set of electrodes and the sets of electrodes are positioned in an electrically insulated holder so that the first set of electrodes is arranged in a non-linear array where one or more of the electrodes is offset from the other electrodes in the array, the second set of electrodes is arranged in a non-linear array where one or more of the electrodes is offset from the other electrodes in the array, and the at least a third set of electrodes is arranged in a non-linear array where one or more of the electrodes is offset from the other electrodes in the array.

14. The ablation apparatus claim 9, wherein the RF power comprises a switching circuit to allow sequential switching of current flow between the at least three sets of electrodes.

15. The ablation apparatus of claim 1, wherein the sets of electrodes are movable between a collapsed position and an expanded position.

16. The ablation apparatus of claim 15, wherein the sets of electrodes movable between a collapsed position and an expanded position are arranged in hollow tube so that the sets of electrodes are collapsed when in the tube and expand when moved outside the tube.

17. The ablation apparatus of claim 15, wherein the hollow tube is a trocar.

18. The ablation apparatus of claim 16, wherein the hollow tube is a stent that is insertable into a luminal space in the body of a subject.

19. An ablation apparatus comprising:
a radio frequency (RF) power source;
a first set of two or more electrodes having tissue piercing distal portions, the first set of electrodes electrically connected via the RF power source;
a second set of two or more electrodes having a tissue piercing distal portions, the second set of electrodes electrically connected via the RF power source; and
a hollow tube,
wherein the sets of electrode movable between a collapsed position and an expanded position and are arranged in the hollow tube so that the sets of electrodes are collapsed when in the tube and expand when moved to a position outside of the tube.

20. A method of tissue ablation comprising:
providing a radio frequency (RF) power source, a first set of two or more electrodes having tissue piercing distal portions, the first set of electrodes electrically connected via the RF power source, and a second set of two or more electrodes having a tissue piercing distal portions, the second set of electrodes electrically connected via the RF power source;
inserting said first and second set of electrodes into a tissue to be ablated;
applying alternating current power via said RF power source so that a current flows from that set electrodes to the other of the first and second set of electrodes thereby creating a zone of ablated tissue.
